(19) United States
(12) Patent Application Publication
(10) Pub. No.: US 2021/0401508 A1
(43) Pub. Date: Dec. 30, 2021

(54) GRAPHICAL USER INTERFACE FOR DEFINING AN ANATOMICAL BOUNDARY

(71) Applicant: Intuitive Surgical Operations, Inc., Sunnyvale, CA (US)

(72) Inventors: Tao Zhao, Sunnyvale, CA (US); Energy Cruse, II, Foster City, CA (US); Vincent Duindam, San Francisco, CA (US); Pechin Chien Pau Lo, Santa Clara, CA (US); Jun Ma, Santa Clara, CA (US); Oscar G. Salazar, Sunnyvale, CA (US); Bai Wang, Palo Alto, CA (US); Hui Zhang, San Jose, CA (US)

(21) Appl. No.: 17/277,414

(22) PCT Filed: Sep. 30, 2019
(86) PCT No.: PCT/US19/53820

§ 371 Date: Mar. 18, 2021

Related U.S. Application Data
(60) Provisional application No. 62/741,157, filed on Oct. 4, 2018.

(51) Int. Cl. A61B 34/20; A61B 34/00; G06T 3/00; A61B 6/00; A61B 34/10

(52) U.S. Cl. CPC A61B 34/20; A61B 34/25; A61B 34/76; G06T 3/0081; A61B 6/463; A61B 2034/107; A61B 34/10; A61B 6/467; A61B 6/5235; A61B 2034/2065; A61B 6/487

(57) ABSTRACT

A medical system may comprise a display system, a user input device, a medical instrument, and a manipulator assembly configured to support and operate the medical instrument. The medical system may also comprise a control system. The control system may be configured to display image data corresponding to a three-dimensional anatomical region via the display system, receive a first user input to generate a first curve in the three-dimensional anatomical region via the user input device, and receive a second user input to generate a second curve in the three-dimensional anatomical region via the user input device. The control system may also be configured to determine an anatomical boundary bounded by the first curve and the second curve. The control system may also be configured to control the manipulator assembly to operate medical instrument using the determined anatomical boundary to limit movement of the medical instrument.

### CROSS-REFERENCE TO RELATED APPLICATIONS

This application claims the benefit of U.S. Provisional Application 62/741,157 filed Oct. 4, 2018, which is incorporated by reference herein in its entirety.

### TECHNICAL FIELD

The present disclosure is directed to systems and methods for planning and performing an image-guided procedure and more particularly to systems and methods for defining an anatomical boundary using a graphical user interface.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions clinicians may insert minimally invasive medical instruments (including surgical, diagnostic, therapeutic, or biopsy instruments) to reach a target tissue location. One such minimally invasive technique is to use a flexible elongate device, such as a catheter, which may be steerable, that can be inserted into anatomic passageways and navigated toward a region of interest within the patient anatomy. Control of such an elongate device by medical personnel during an image-guided procedure involves the management of several degrees of freedom including at least the management of insertion and retraction of the elongate device as well as steering or bend radius of the device. In addition, different modes of operation may also be supported.

Accordingly, it would be advantageous to provide a graphical user interface that supports intuitive planning of medical procedures including minimally invasive medical techniques.

### SUMMARY

The embodiments of the invention are best summarized by the claims that follow the description.

In one embodiment, a medical system comprises a display system and a user input device. The medical system also comprises a control system communicatively coupled to the display system and the user input device. The control system is configured to display image data corresponding to a three-dimensional anatomical region via the display system and receive a first user input to generate a first curve in the three-dimensional anatomical region via the user input device. The control system is also configured to receive a second user input to generate a second curve in the three-dimensional anatomical region via the user input device and determine an anatomical boundary bounded by the first curve and the second curve. The anatomical boundary indicates a surface of an anatomical structure in the three-dimensional anatomical region.

In another embodiment, a method of planning a medical procedure comprises displaying, via a display system, image data corresponding to a three-dimensional anatomical region and receiving, via a user input device, a plurality of user inputs to generate a plurality of curves in the three-dimensional anatomical region. The method also comprises determining from the plurality of curves an anatomical boundary. The anatomical boundary demarcates a vulnerable portion of the three-dimensional anatomical region. The method also comprises displaying, via the display system, the anatomical boundary overlaid on the image data.

In another embodiment, a non-transitory machine-readable medium comprises a plurality of machine readable instructions which when executed by one or more processors associated with a planning workstation are adapted to cause the one or more processors to perform a method. The method comprises displaying, via a display system, CT image data corresponding to a lung and receiving, via a user input device, a plurality of user inputs to generate a plurality of curves in different slices of the CT image data. The method also includes interpolating among the plurality of curves to determine an anatomical boundary indicating a location of a pleura of the lung in the CT image data; and displaying, via the display system, the anatomical boundary overlaid on the CT image data.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

The patent or application file contains at least one drawing executed in color. Copies of this patent or patent application publication with color drawing(s) will be provided by the Office upon request and payment of the necessary fee.

FIG. 1 is a simplified diagram of a medical system according to some embodiments.

FIGS. 2A and 2B are simplified diagrams of side views of a patient coordinate space including a medical instrument mounted on an insertion assembly according to some embodiments.

FIG. 3A is a simplified diagram of a method for defining an anatomical boundary according to some embodiments.

FIG. 3B is a diagram of a method for defining an anatomical boundary according to other embodiments.

FIG. 3C is a diagram of a method for presenting an anatomical boundary according to some embodiments.

FIG. 3D is a diagram of a method for providing guidance information to guide the determination of an anatomical boundary according to some embodiments.

FIGS. 3E-3G are diagrams of methods for providing guidance information according to some embodiments.

FIGS. 4A-4F are simplified diagrams of a graphical user interface during the performance of the method for defining an anatomical boundary according to some embodiments.

FIGS. 5A and 5B are simplified diagrams illustrating range guidance associated with an anatomical boundary according to some embodiments.

FIG. 5C illustrates a graphical user interface that presents range guidance with the two-dimensional image data.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

In some instances well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

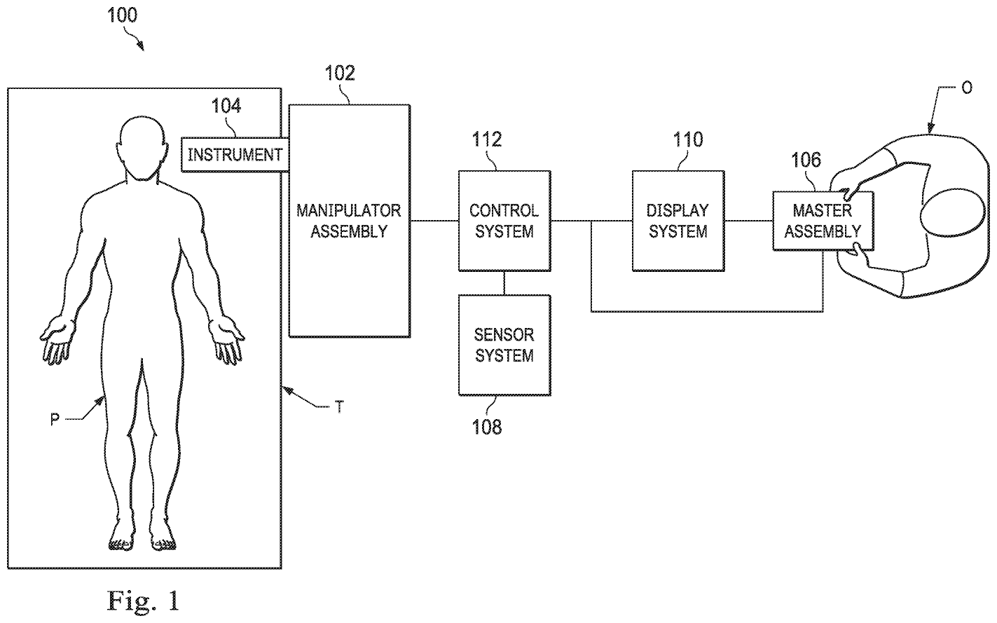

As shown in FIG. 1, medical system 100 generally includes a manipulator assembly 102 for operating a medical instrument 104 in performing various procedures on a patient P. Medical instrument 104 may extend into an internal surgical site within the body of patient P via an opening in the body of patient P. The medical system 100 may be teleoperated, non-teleoperated, or a hybrid of the two. The manipulator assembly 102 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. Manipulator assembly 102 is mounted to or near an operating table T. A master assembly 106 allows an operator O (e.g., a surgeon, a clinician, or a physician as illustrated in FIG. 1) to view the interventional site and to control manipulator assembly 102.

Master assembly 106 may be located at an operator console which is usually located in the same room as operating table T, such as at the side of a surgical table on which patient P is located. However, it should be understood that operator O can be located in a different room or a completely different building from patient P. Master assembly 106 generally includes one or more control devices for controlling manipulator assembly 102. The control devices may include any number of a variety of input devices, such as joysticks, trackballs, data gloves, trigger-guns, hand-operated controllers, voice recognition devices, body motion or presence sensors, and/or the like.

Manipulator assembly 102 supports medical instrument 104 and may include a kinematic structure of one or more non-servo controlled links (e.g., one or more links that may be manually positioned and locked in place, generally referred to as a set-up structure), and/or one or more servo controlled links (e.g. one more links that may be controlled in response to commands from the control system), and a manipulator. Manipulator assembly 102 may optionally include a plurality of actuators or motors that drive inputs on medical instrument 104 in response to commands from the control system (e.g., a control system 112). The actuators may optionally include drive systems that when coupled to medical instrument 104 may advance medical instrument 104 into a naturally or surgically created anatomic orifice. Other drive systems may move the distal end of medical instrument 104 in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to actuate an articulable end effector of medical instrument 104 for grasping tissue in the jaws of a biopsy device and/or the like.

Medical system 100 may include a sensor system 108 with one or more sub-systems for receiving information about the manipulator assembly 102 and/or the medical instrument 104. Such sub-systems may include a position/location sensor system (e.g., an electromagnetic (EM) sensor system); a shape sensor system for determining the position, orientation, speed, velocity, pose, and/or shape of a distal end and/or of one or more segments along a flexible body that may make up medical instrument 104; a visualization system for capturing images from the distal end of medical instrument 104; and actuator position sensors such as resolvers, encoders, potentiometers, and the like that describe the rotation and orientation of the motors controlling the instrument 104.

Medical system 100 also includes a display system 110 for displaying an image or representation of the surgical site and medical instrument 104. Display system 110 and master assembly 106 may be oriented so operator O can control medical instrument 104 and master assembly 106 with the perception of telepresence.

In some embodiments, medical instrument 104 may include a visualization system which may include an image capture assembly that records a concurrent or real-time images of a surgical site and provides the image to the operator O through one or more displays of display system 110. The concurrent image may be, for example, a two or three dimensional image captured by an endoscope positioned within the surgical site. In some embodiments, the visualization system includes endoscopic components that may be integrally or removably coupled to medical instrument 104. However in some embodiments, a separate endoscope, attached to a separate manipulator assembly may be used with medical instrument 104 to image the surgical site. The visualization system may be implemented as hardware, firmware, software or a combination thereof which interact with or are otherwise executed by one or more computer processors, which may include the processors of a control system 112.

Display system 110 may also display an image of the surgical site and medical instruments captured by the visualization system. In some examples, medical system 100 may configure medical instrument 104 and controls of master assembly 106 such that the relative positions of the medical instruments are similar to the relative positions of the eyes and hands of operator O. In this manner operator O can manipulate medical instrument 104 and the hand control as if viewing the workspace in substantially true presence. By true presence, it is meant that the presentation of an image is a true perspective image simulating the viewpoint of a physician that is physically manipulating medical instrument 104.

In some examples, display system 110 may present images of a surgical site recorded pre-operatively or intra-operatively using image data from imaging technology such as, computed tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The pre-operative or intra-operative image data may be presented as two-dimensional, three-dimensional, or four-dimensional (including e.g., time based or velocity based information) images and/or as images from models created from the pre-operative or intra-operative image data sets.

In some embodiments, often for purposes of image-guided medical procedures, display system 110 may display a virtual navigational image in which the actual location of medical instrument 104 is registered (i.e., dynamically referenced) with the preoperative or concurrent images/model. This may be done to present the operator O with a virtual image of the internal surgical site from a viewpoint of medical instrument 104.

Medical system 100 may also include control system 112. Control system 112 includes at least one memory and at least one computer processor (not shown) for effecting control between medical instrument 104, master assembly 106, sensor system 108, and display system 110. Control system 112 also includes programmed instructions (e.g., a non-transitory machine-readable medium storing the instructions) to implement some or all of the methods described in accordance with aspects disclosed herein, including instructions for providing information to display system 110. While control system 112 is shown as a single block in the simplified schematic of FIG. 1, the system may include two or more data processing circuits with one portion of the processing optionally being performed on or adjacent to manipulator assembly 102, another portion of the processing being performed at master assembly 106, and/or the like. The processors of control system 112 may execute instructions comprising instruction corresponding to processes disclosed herein and described in more detail below. In some embodiments, control system 112 may receive force and/or torque feedback from medical instrument 104. Responsive to the feedback, control system 112 may transmit signals to master assembly 106. In some examples, control system 112 may transmit signals instructing one or more actuators of manipulator assembly 102 to move medical instrument 104.

Control system 112 may optionally further include a virtual visualization system to provide navigation assistance to operator O when controlling medical instrument 104 during an image-guided medical procedure. Virtual navigation using the virtual visualization system may be based upon reference to an acquired preoperative or intraoperative dataset of anatomic passageways. Software, which may be used in combination with operator inputs, is used to convert the recorded images into segmented two dimensional or three dimensional composite representation of a partial or an entire anatomic organ or anatomic region. An image data set is associated with the composite representation. The virtual visualization system obtains sensor data from sensor system 108 that is used to compute an approximate location of medical instrument 104 with respect to the anatomy of patient P. The system may implement the sensor system 108 to register and display the medical instrument together with the preoperatively or intraoperatively recorded surgical images. For example, PCT Publication WO 2016/191298 (published Dec. 1, 2016) (disclosing “Systems and Methods of Registration for Image Guided Surgery”), which is incorporated by reference herein in its entirety, discloses such one system.

Medical system 100 may further include optional operations and support systems (not shown) such as illumination systems, steering control systems, irrigation systems, and/or suction systems. In some embodiments, medical system 100 may include more than one manipulator assembly and/or more than one master assembly. The exact number of manipulator assemblies will depend on the medical procedure and the space constraints within the operating room, among other factors. Master assembly 106 may be collocated or they may be positioned in separate locations. Multiple master assemblies allow more than one operator to control one or more manipulator assemblies in various combinations.

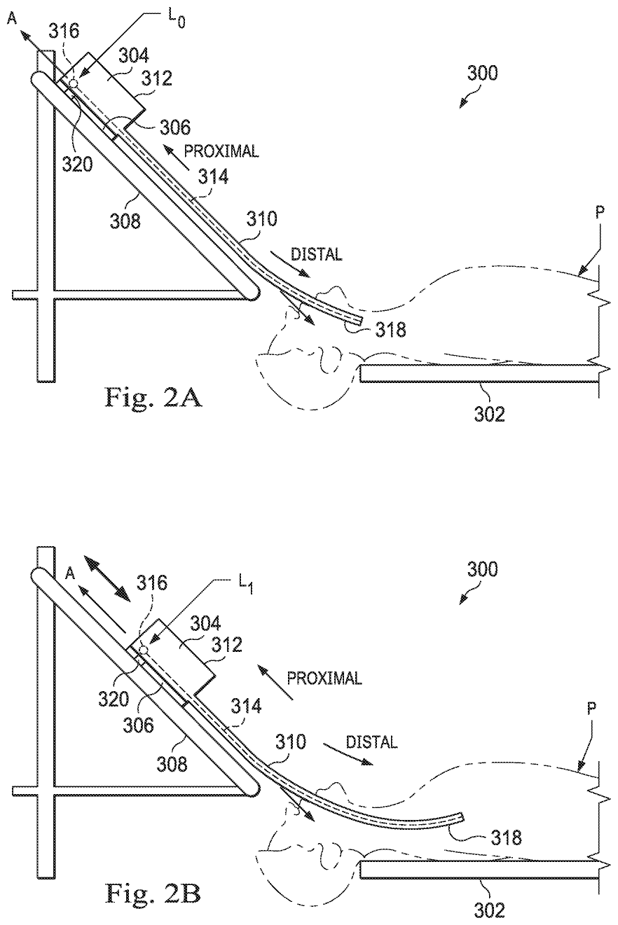

FIGS. 2A and 2B are simplified diagrams of side views of a patient coordinate space including a medical instrument mounted on an insertion assembly according to some embodiments. As shown in FIGS. 2A and 2B, a surgical environment 300 including a patient P is positioned on the table T of FIG. 1. Patient P may be stationary within the surgical environment in the sense that gross patient movement is limited by sedation, restraint, and/or other means. Cyclic anatomic motion including respiration and cardiac motion of patient P may continue. Within surgical environment 300, a medical instrument 304 is used to perform a medical procedure which may include, for example, surgery, biopsy, ablation, illumination, irrigation, suction, or a system registration procedure. The medical instrument 304 may be, for example, the instrument 104. The instrument 304 includes a flexible elongate device 310 (e.g., a catheter) coupled to an instrument body 312. Elongate device 310 includes one or more channels of shown) sized and shaped to receive a medical tool (not shown).

Elongate device 310 may also include one or more sensors (e.g., components of the sensor system 108). In some embodiments, an optical fiber shape sensor 314 is fixed at a proximal point 316 on instrument body 312. In some embodiments, proximal point 316 of optical fiber shape sensor 314 may be movable along with instrument body 312 but the location of proximal point 316 may be known (e.g., via a tracking sensor or other tracking device). Shape sensor 314 measures a shape from proximal point 316 to another point such as distal end 318 of elongate device 310. Shape sensor 314 may be aligned with flexible elongate device 310 (e.g., provided within an interior channel (not shown) or mounted externally). In one embodiment, the optical fiber has a diameter of approximately 200 μm. In other embodiments, the dimensions may be larger or smaller. The shape sensor 314 may be used to determine the shape of flexible elongate device 310. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. patent application Ser. No. 11/180,389 (filed Jul. 13, 2005) (disclosing “Fiber optic position and shape sensing device and method relating thereto”); U.S. patent application Ser. No. 12/047,056 (filed on Jul. 16, 2004) (disclosing “Fiber-optic shape and relative position sensing”); and U.S. Pat. No. 6,389,187 (filed on Jun. 17, 1998) (disclosing “Optical Fibre Bend Sensor”), which are all incorporated by reference herein in their entireties. Sensors in some embodiments may employ other suitable strain sensing techniques, such as Rayleigh scattering, Raman scattering, Brillouin scattering, and fluorescence scattering. Various systems for using fiber optic sensors to register and display a surgical instrument with surgical images are provided in PCT Publication WO 2016/191298 (published Dec. 1, 2016) (disclosing “Systems and Methods of Registration for Image Guided Surgery”), which is incorporated by reference herein in its entirety.

In various embodiments, position sensors such as electromagnetic (EM) sensors, may be incorporated into the medical instrument 304. In various embodiments, a series of position sensors may be positioned along elongate device 310 and then used for shape sensing. In some embodiments, position sensors may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point. Further description of a position sensor system is provided in U.S. Pat. No. 6,380,732 (filed Aug. 11, 1999) (disclosing “Six-Degree of Freedom Tracking System Having a Passive Transponder on the Object Being Tracked”), which is incorporated by reference herein in its entirety.

Elongate device 310 may also house cables, linkages, or other steering controls (not shown) that extend between instrument body 312 and distal end 318 to controllably bend distal end 318. In some examples, at least four cables are used to provide independent “up-down” steering to control a pitch of distal end 318 and “left-right” steering to control a yaw of distal end 318. Steerable elongate devices are described in detail in U.S. patent application Ser. No. 13/274,208 (filed Oct. 14, 2011) (disclosing “Catheter with Removable Vision Probe”), which is incorporated by reference herein in its entirety. The instrument body 312 may include drive inputs that removably couple to and receive power from drive elements, such as actuators, of the manipulator assembly.

Instrument body 312 may be coupled to instrument carriage 306. Instrument carriage 306 is mounted to an insertion stage 308 fixed within surgical environment 300. Alternatively, insertion stage 308 may be movable but have a known location (e.g., via a tracking sensor or other (racking device) within surgical environment 300. Instrument carriage 306 may be a component of a manipulator assembly (e.g., manipulator assembly 102) that couples to medical instrument 304 to control insertion motion (i.e., motion along the A axis) and, optionally, motion of a distal end 318 of an elongate device 310 in multiple directions including yaw, pitch, and roll. Instrument carriage 306 or insertion stage 308 may include actuators, such as servomotors, (not shown) that control motion of instrument carriage 306 along insertion stage 308.

A sensor device 320, which may be a component of the sensor system 108, provides information about the position of instrument body 312 as it moves on insertion stage 308 along an insertion axis A. Sensor device 320 may include resolvers, encoders, potentiometers, and/or other sensors that determine the rotation and/or orientation of the actuators controlling the motion of instrument carriage 306 and consequently the motion of instrument body 312. In some embodiments, insertion stage 308 is linear. In some embodiments, insertion stage 308 may be curved or have a combination of curved and linear sections.

FIG. 2A shows instrument body 312 and instrument carriage 306 in a retracted position along insertion stage 308. In this retracted position, the proximal point 316 is at a position L₀ on axis A. In this position along insertion stage 308, the location of proximal point 316 may be set to a zero and/or another reference value to provide a base reference to describe the position of instrument carriage 306, and thus proximal point 316, on insertion stage 308. With this retracted position of instrument body 312 and instrument carriage 306, distal end 318 of elongate device 310 may be positioned just inside an entry orifice of patient P. Also in this position, sensor device 320 may be set to a zero and/or another reference value (e.g., I=0). In FIG. 2B, instrument body 312 and instrument carriage 306 have advanced along the linear track of insertion stage 308 and distal end 318 of elongate device 310 has advanced into patient P. In this advanced position, the proximal point 316 is at a position L₁ on the axis A. In some examples, encoder and/or other position data from one or more actuators controlling movement of instrument carriage 306 along insertion stage 308 and/or one or more position sensors associated with instrument carriage 306 and/or insertion stage 308 is used to determine the position Lₓ of proximal point 316 relative to position L₀. In some examples, position L may further be used as an indicator of the distance or insertion depth to which distal end 318 of elongate device 310 is inserted into the passageways of the anatomy of patient P.

In an illustrative application, a medical system, such as medical system 100, may include a robotic catheter system for use in lung biopsy procedures. A catheter of the robotic catheter system provides a conduit for tools such as endoscopes, endobronchial ultrasound (EBUS) probes, and/or biopsy tools to be delivered to locations within the airways where one or more anatomic targets of the lung biopsy, such as lesions, nodules, tumors, and/or the like, are present. When the catheter is driven through anatomy, typically an endoscope is installed such that a clinician, such as surgeon O, can monitor a live camera feed of a distal end of the catheter. The live camera feed and/or other real-time navigation information may be displayed to the clinician via a graphical user interface. An example of a graphical user interface for monitoring the biopsy procedure is covered in U.S. Provisional Patent Application No. 62/486,879 entitled “Graphical User Interface for Monitoring an Image-Guided Procedure and filed Apr. 18, 2017, which is hereby incorporated by reference in its entirety.

Before the biopsy procedure is performed using the robotic catheter system, pre-operative planning steps may be performed to plan the biopsy procedure. Pre-operative planning steps may include segmentation of image data, such as a patient CT scan, to create a 3D model of anatomy, selecting anatomic targets within the 3D model, determining airways in the model, growing the airways to form a connected tree of airways, and planning trajectories between the targets and the connected tree. One or more of these steps may be performed on the same robotic catheter system used to perform the biopsy. Alternately or additionally, planning may be performed on a different system, such as a workstation dedicated to pre-operative planning. The plan for the biopsy procedure may be saved (e.g., as one or more digital files) and transferred to the robotic catheter system used to perform the biopsy procedure. The saved plan may include the 3D model, identification of airways, target locations, trajectories to target locations, routes through the 3D model, and/or the like.

Illustrative embodiments of a graphical user interface for planning a medical procedure, including but not limited to the lung biopsy procedure described above, are provided below. The graphical user interface may include a plurality of modes including a data selection mode, a hybrid segmentation and planning mode, a preview mode, a save mode, a management mode, and a review mode. Some aspects of the graphical user interface are similar to features described in U.S. Provisional Patent Application No. 62/357,217, entitled “Graphical User Interface for Displaying Guidance Information During and Image-Guided. Procedure” and filed Jun. 30, 2016, and U.S. Provisional Patent Application No. 62/357,258, entitled “Graphical User Interface for Displaying Guidance Information in a Plurality of Modes During and Image-Guided Procedure” and filed Jun. 30, 2016, which are hereby incorporated by reference in their entirety.

In the planning and execution of a medical procedure, an anatomical boundary or a virtual “hazard fence” may be defined by identifying a surface that is not to be crossed by a medical instrument during the medical procedure. The anatomical boundary may shield vulnerable portions of the anatomy that are in the vicinity of the target location or other portions of interest from being inadvertently penetrated by the medical instrument. Portions of interest, including vulnerable anatomic structures or surfaces, may include, for example, pulmonary pleurae, pulmonary fissures, large bullae, and blood vessels. For example, puncturing the lung pleura during the medical procedure could cause dangerous pneumothorax to the patient. Consistent with such embodiments, defining an anatomical boundary corresponding to the lung pleura may allow the operator to constrain the path of the medical instrument to avoid the vulnerable portion of the anatomy. For example, a candidate path may be invalid when it passes within a threshold distance of a vulnerable portion of the anatomy, breaches a vulnerable portion of the anatomy, and/or the like.

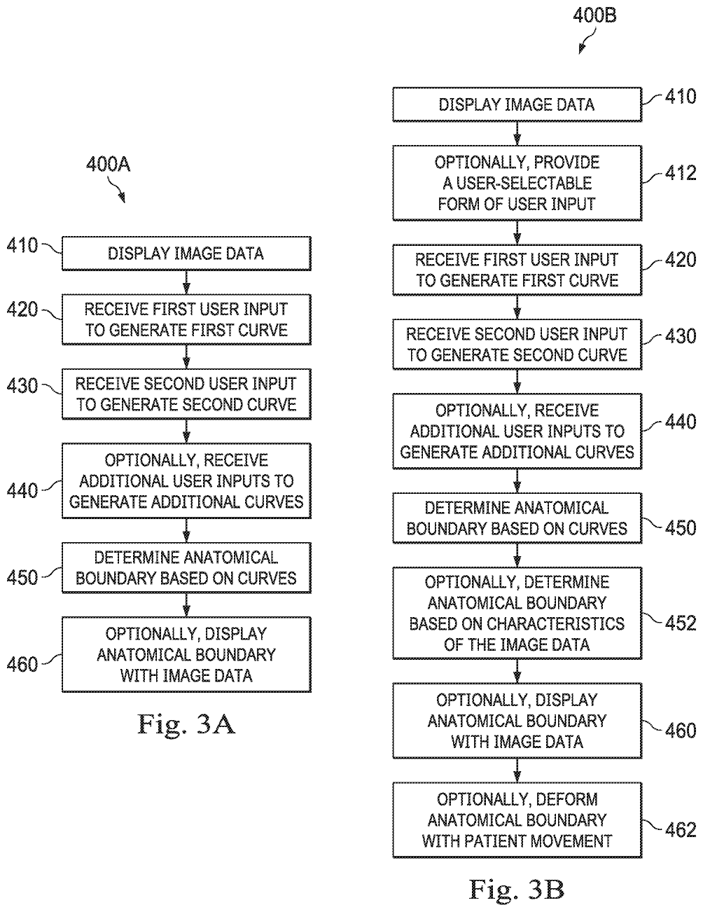

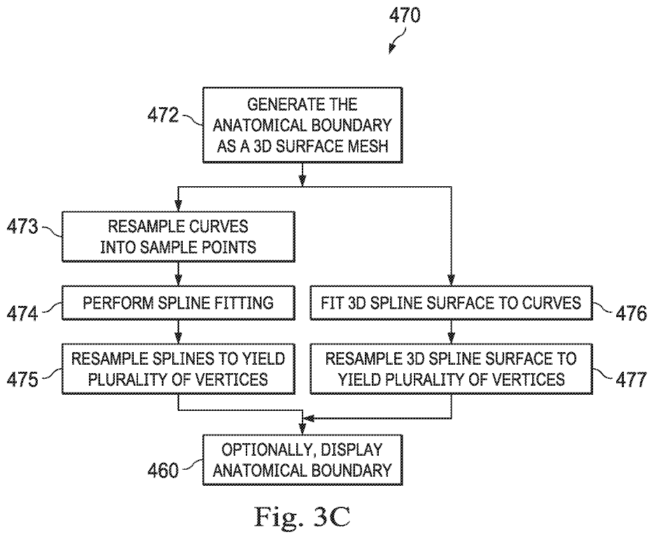

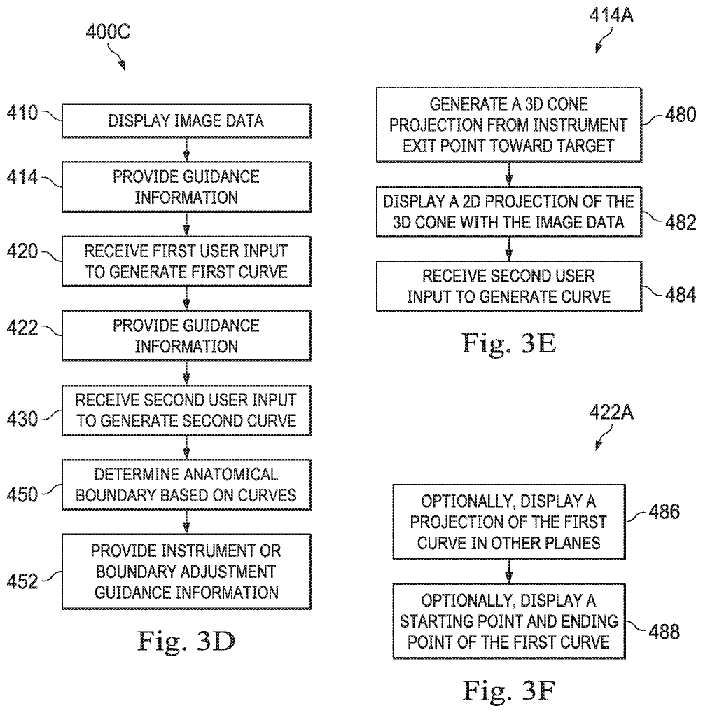

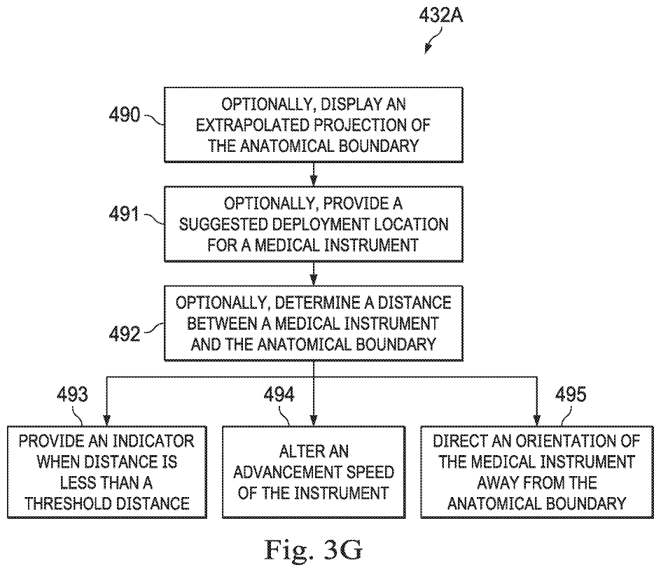

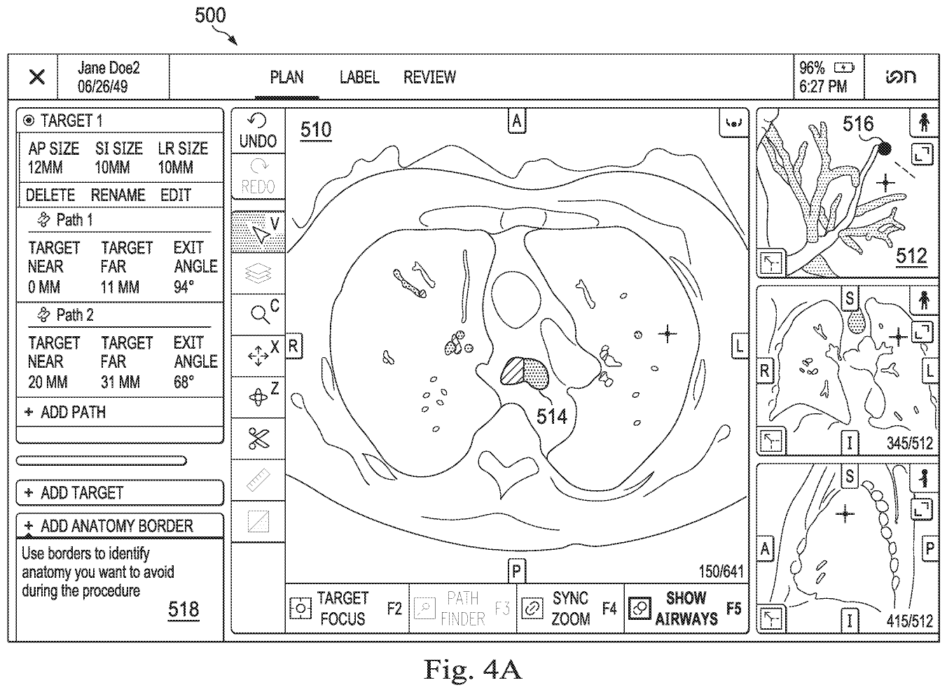

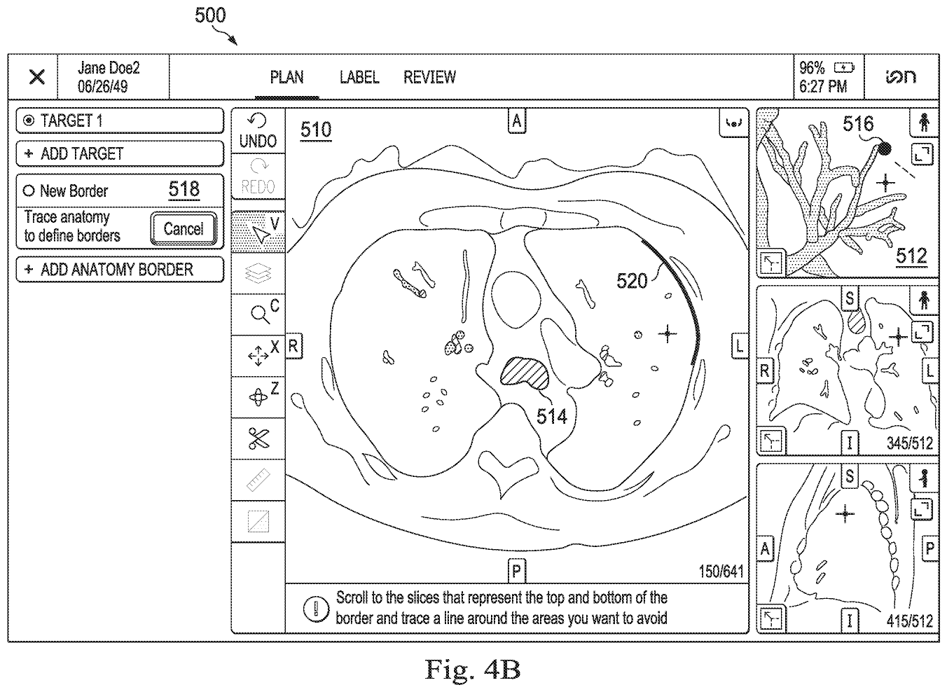

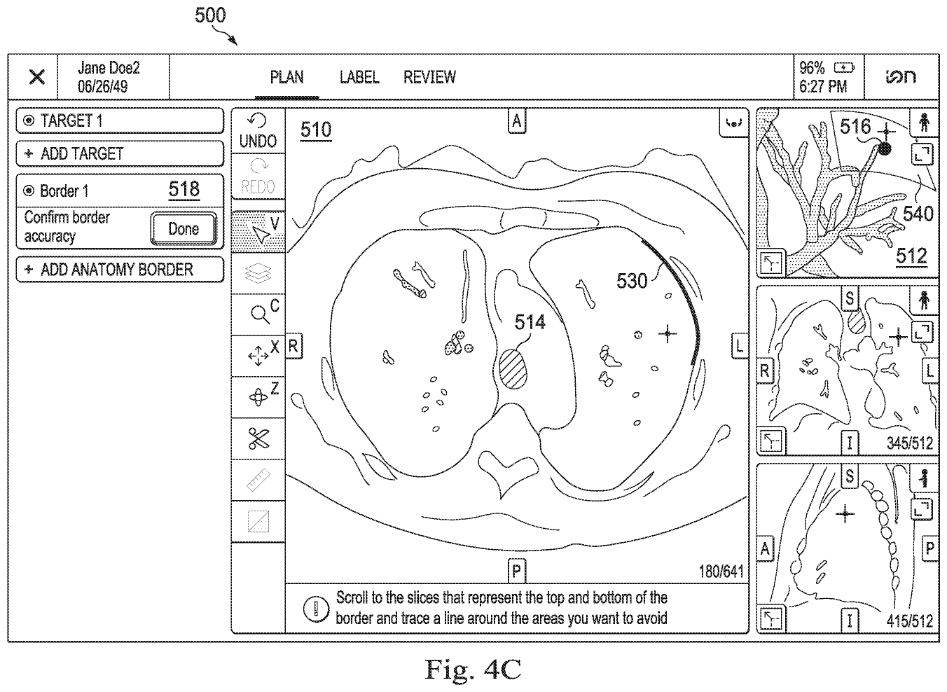

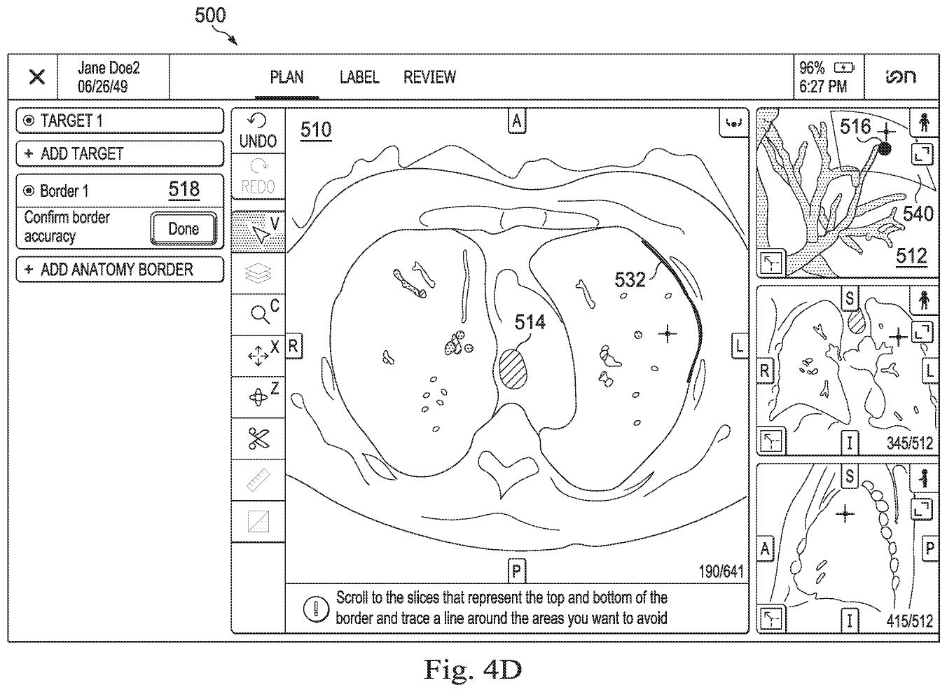

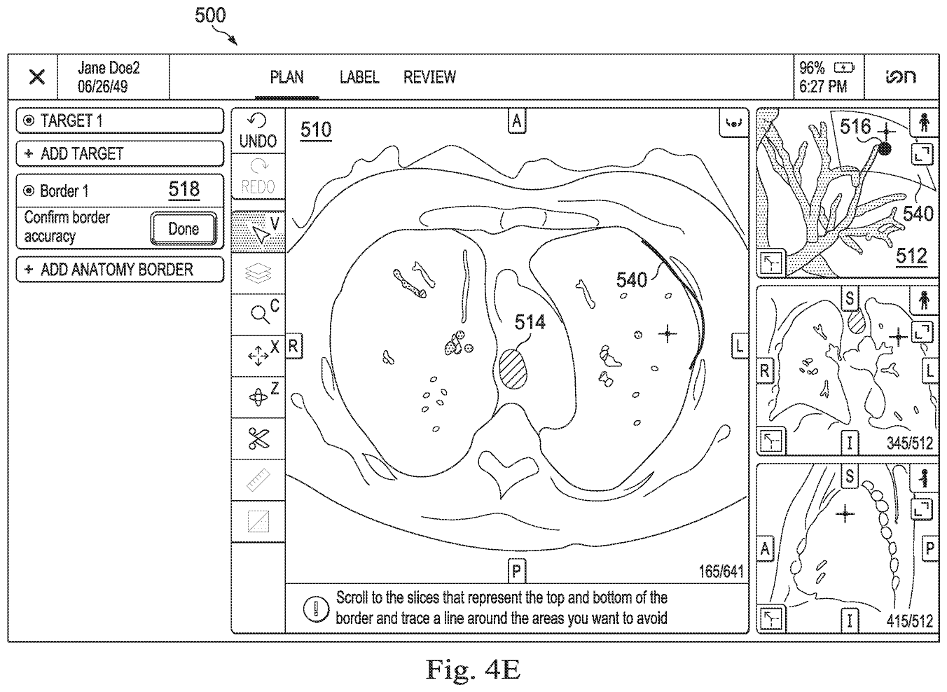

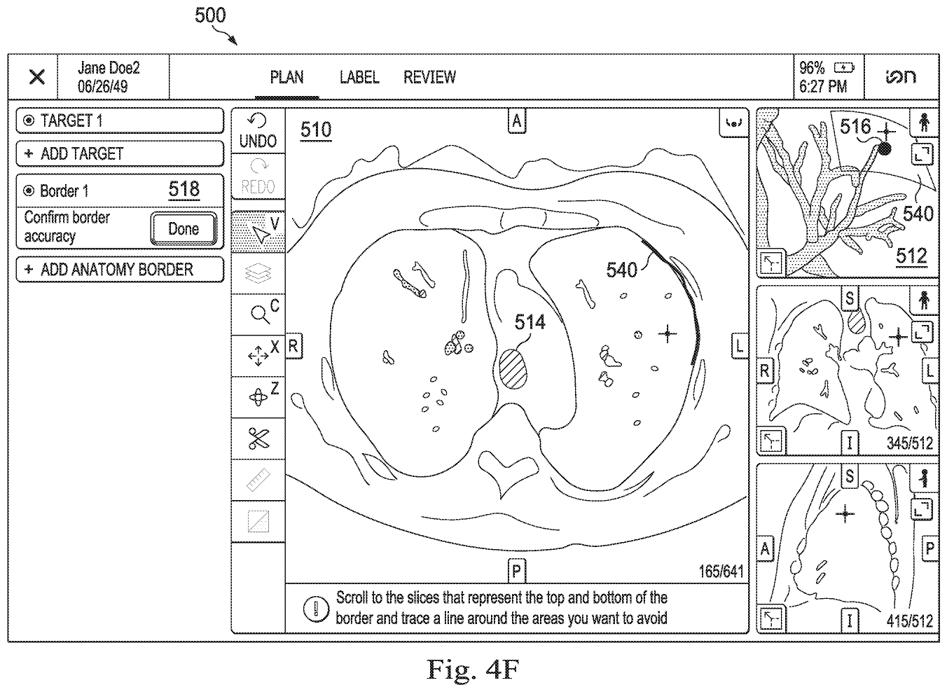

FIG. 3A is a simplified diagram of a method 400A for defining an anatomical boundary according to some embodiments. FIGS. 4A-4F are corresponding simplified diagrams of a graphical user interface 500 during the performance of method 400A according to some embodiments. In some embodiments consistent with FIGS. 1-2B, graphical user interface 500 may be displayable on a display system, such as display system 110 and/or a display system of an independent planning workstation.

Graphical user interface 500 displays information associated with planning a medical procedure in one or more views that are viewable to a user, such as operator O. Although illustrative arrangements of views are depicted in FIGS. 4A-4F, it is to be understood that graphical user interface 500 may display any suitable number of views, in any suitable arrangement, and/or on any suitable number of screens. In some examples, the number of concurrently displayed views may be varied by opening and closing views, minimizing and maximizing views, moving views between a foreground and background of graphical user interface 500, switching between screens, and/or otherwise fully or partially obscuring views. Similarly, the arrangement of the views—including their size, shape, orientation, ordering (in a case of overlapping views), nd/or the like—may vary and/or may be user-configurable.

The methods disclosed herein are illustrated as a set of operations or processes. Not all of the illustrated processes may be performed in all embodiments of an illustrated method. Additionally, one or more processes that are not expressly illustrated may be included before, after, in between, or as part of the illustrated processes. In some embodiments, one or more of the processes may be implemented, at least in part, in the form of executable code stored on non-transitory, tangible, machine-readable media that when run by one or more processors (e.g., the processors of a control system) may cause the one or more processors to perform one or more of the processes. In one or more embodiments, the processes may be performed by the control system 112.

At a process 410, image data 510 corresponding to a three-dimensional anatomical region of a patient P is displayed via graphical user interface 500. As depicted in FIGS. 4A-4F, image data 510 may include, for example computed tomography (CT) image data. The image data 510 may include multiple images of the three-dimensional anatomical region with FIG. 4A illustrating a single plane or “slice” of the image data. Additionally or alternately, image data 510 may include a 3D anatomical model, as depicted in a thumbnail view 512 of graphical user interface 500. In some embodiments, image data 510 may include segmentation data 514 that indicates the location of anatomical features identified from the CT image data, such as airways in the lungs, blood vessels, or the like. In some embodiments, image data 510 may include an anatomic target 516 of the medical procedure, such as a biopsy site. In various alternative embodiments, image data may be generated using other imaging technologies such as magnetic resonance imaging (MRI), fluoroscopy, thermography, ultrasound, optical coherence tomography (OCT), thermal imaging, impedance imaging, laser imaging, nanotube X-ray imaging, and/or the like.

At a process 420, a first user input for generating or defining a curve 520 in the three-dimensional anatomical region is received via a user input device. The curve 520 is generated in one plane of the image data 510. In some embodiments, the first user input may be provided by the operator via a mouse, a touchscreen, a stylus, or the like. As depicted in FIG. 413, curve 520 may be displayed via graphical user interface 500. In this embodiment, the curve 520 may correspond to a surface identified by the operator as a portion of the pulmonary pleura.

At a process 430, a second user input for generating or defining a second curve 530 in the three-dimensional anatomical region is received via the user input device. The curve 530 is generated in a plane of the image data 510 that is different from the image plan in which the curve 520 was defined. As depicted in FIG. 4C, the curve 530 may be displayed via graphical user interface 500.

At a process 440, optionally, additional user inputs may be received, each additional user input generating or defining an additional curve (e.g., additional curve 532, FIG. 4D) in the three-dimensional anatomical region. In general, curves 530, 532 and any additional curves are defined in a similar manner to curve 520. Any additional curves may be positioned in different planes of image data 510 (e.g., in a different slice of the CT image data) relative to curves 520, 530 and to each other in any order.

At a process 450 and as illustrated in FIG. 4E, an anatomical boundary 540 is determined that is bounded by curve 520, curve 530, and any additional curves. In some embodiments, the anatomical boundary is determined by interpolating or otherwise identifying intermediate curves that bound the boundary 540. According to some embodiments, anatomical boundary 540 may indicate a surface of the three-dimensional anatomical region or surface that, is vulnerable, or otherwise of interest, and is not to be crossed by a medical instrument during the medical procedure.

Optionally, at a process 460, anatomical boundary 540 is displayed via graphical user interface 500. According to some embodiments, a visual representation of anatomical boundary 540 may be overlaid on the image data. As depicted in FIGS. 4E and 4F, a cross sectional representation of anatomical boundary 540 may be displayed as a curve overlaid on a CT slice, a three dimensional representation of anatomical boundary 540 may be displayed as a translucent or grid-wire mesh on the 3D anatomical model in thumbnail view 512, or the like.

In some cases, an interpolated portion of anatomical boundary 540 may not accurately track the actual anatomical boundary that the operator seeks to define. For example, in the illustrative example depicted in FIG. 4E, an interpolated portion of anatomical boundary 540 between curve 520 and curve 530, which is intended to track the pleura of the lungs, is visibly misaligned from the pleura. To correct this misalignment, method 400A may return to processes 420-460 to receive additional user inputs defining additional curves that are used to update anatomical boundary 540 to more closely align with the desired anatomical boundary (as depicted in FIG. 4F). In this manner, processes 420-450 may be performed iteratively until satisfactory alignment is achieved. In a similar manner, the range of anatomical boundary 540 may be extended by returning to processes 420-450 to receive additional user inputs defining additional curves that are outside the current range of anatomical boundary 540.

FIG. 3B is a diagram of a method 400B for defining an anatomical boundary according to some embodiments. Some processes in method 400B are the same as those identified in FIG. 400A and are indicated with the same reference numeral.

Before or after the display of image data at process 410, at an optional process 412, the user may be presented with a selectable choice between curve drawing options including freehand and polyline form. In some embodiments, curve 520 may be drawn in freehand, in polyline form, in a series of plotted points, or the like. In the case of a polyline input (e.g. a series of straight line segments) or the series of plotted points, the curve 520 may be determined, for example, by spline fitting. Optionally, the spline fitting may be performed when all the points are received. Optionally, the spline fitting may be performed on all the received points and is updated when a new point is received. Optionally, the spline fitting may be performed by all points that have been received and the current mouse location so that user can see in real time the shape of the fitted curve before a point is received. According to some embodiments, the first user input may be received in response to receiving a selection of an anatomical boundary tool 518 by the operator. The selection of anatomical boundary tool 518 indicates that the operator intends to define an anatomical boundary via graphical user interface 500.

At the process 450, anatomical boundary 540 may be determined based on stored or displayed as a three-dimensional surface mesh that includes a plurality of vertices. FIG. 3C illustrates a method 470 for presenting an anatomical boundary according to some embodiments. At a process 472, anatomical boundary 540 may be generated as a three-dimensional surface mesh that includes a plurality of vertices. In one optional technique, at a process 473 the vertices of the three-dimensional surface mesh may be determined by resampling each of curve 520, curve 530, and any additional curves into an equal number of sample points. At a process 474, spline fitting is performed between matching sample points from the respective curves, yielding a plurality of splines. At a process 475, each of the plurality of splines is resampled to yield the vertices of the three-dimensional surface mesh. In another optional technique, at a process 476, the vertices of the three-dimensional surface mesh may be determined by fitting a three-dimensional spline surface to curve 520, curve 530 and any additional curves. At a process 477, the three-dimensional spline surface is resampled to yield the vertices of the three-dimensional surface mesh.

Referring again to FIG. 3B, at an optional process 452, the anatomical boundary 540 may further be determined based on characteristics of the image data 510. For example, anatomical boundary 540 may be snapped to areas of image data 510 with a high intensity gradient, as a high intensity gradient indicates the presence of a surface of interest (e.g., the pleura of the lungs, a blood vessel wall, etc.). Similarly, computer vision techniques, including machine learning algorithms, may be applied to image data 510 to identify candidate anatomical boundaries. Consistent with such embodiments, anatomical boundary 540 may be snapped to a candidate anatomical boundary determined by such computer vision or machine learning techniques.

At an optional process 462, the anatomical boundary 540 may be deformed based on patient movement. During navigation, the patient anatomy and consequently the model may move or become deformed by, for example, forces from the medical instrument, the lung expiration and inspiration, and the beating heart. The deformation may be measured, for example by a shape sensor in the medical instrument or predicted by simulation and the deformation may be applied to the model. The anatomical boundary 540 may likewise be adjusted or deformed to correspond to the deformation of the model.

FIG. 3D is a diagram of a method 400C for defining an anatomical boundary according to some embodiments. Some processes in method 400C are the same as those identified in FIG. 400A and are indicated with the same reference numeral. At processes 414, 422, and 452 various guidance information and visualization aides may be displayed via graphical user interface 500 to assist the operator in defining or adjusting anatomical boundary 540.

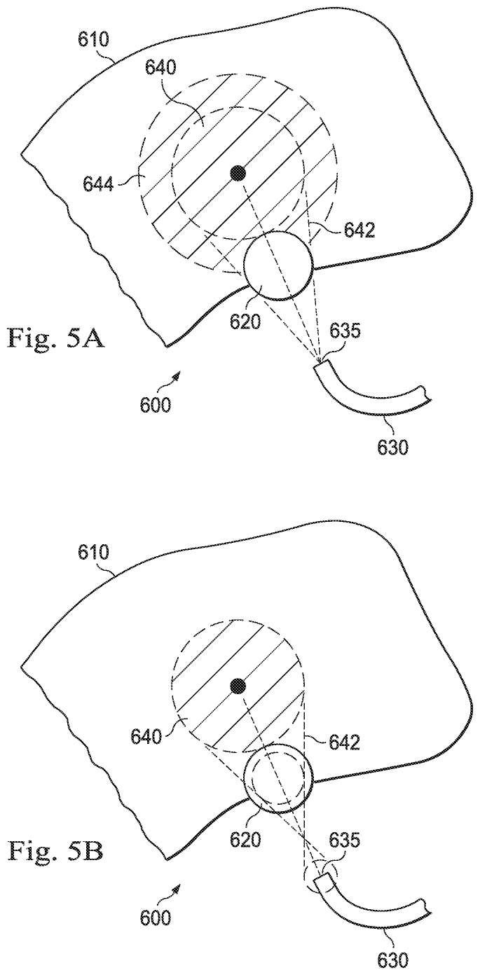

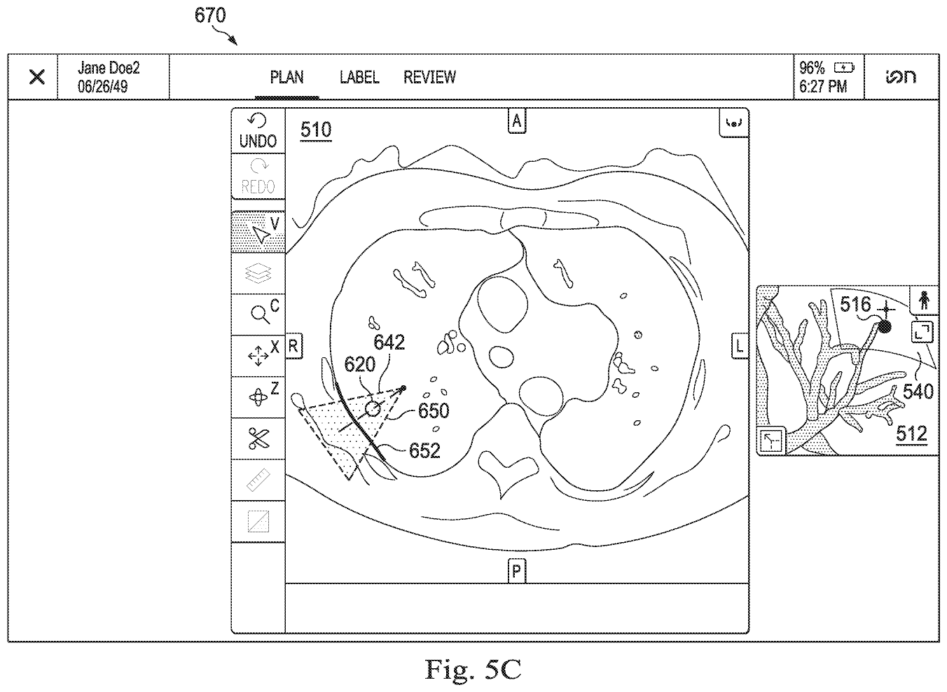

At the process 414, guidance information and visualization aides may further be displayed to suggest a range or shape that anatomical border 540 should cover. Accordingly, range guidance information may be displayed to improve the range of protection provided by anatomical border 540, as discussed in further detail below with reference to FIGS. 5A-5B.

FIGS. 5A and 5B are simplified diagrams illustrating range guidance 600 associated with an anatomical boundary, such as anatomical boundary 540, according to some embodiments. FIG. 5C illustrates a graphical user interface 670 that presents the range guidance 600 with the two-dimensional image data 510 to assist and guide the user in drawing the curves. As discussed previously, anatomical boundary 540 generally identifies a surface 610, such as the lung pleura, that should not be punctured or otherwise contacted or crossed by a medical instrument during a medical procedure at a site of a target 620. As illustratively depicted in FIGS. 5A and 5B, the medical procedure may correspond to a biopsy procedure in which a catheter 630 is inserted into the vicinity of target 620. During the biopsy procedure, a needle is aimed from an exit point 635 of catheter 630 towards target 620. Accordingly, in the biopsy procedure (and various other types of procedures in which instruments may be extended from catheter 630 towards target 620), anatomical boundary 540 may be used to identify the portion of surface 610 that is behind target 620 relative to exit point 635 and therefore at risk of being punctured if the needle (or other instrument) extends too far past target 620.

As depicted in FIG. 5A, a three-dimensional at-risk portion 640 of surface 610 is determined based on an intersection of anatomical surface 610 and a three-dimensional zone. The zone may be, for example, a cone-shaped projection 642 extending from exit point 635 through target 620. In some embodiments, at-risk portion 640 may include an additional margin 644 beyond the region directly within projection 642. In some embodiments, at-risk portion 640 may be determined in a binary manner (e.g., a given portion is either deemed at-risk or not) or in a gradual or continuous manner to reflect varying levels of risk at different locations.

Based on determining the at-risk portion 640 of surface 610, guidance information may be provided in image data 510 to the operator to ensure that the anatomical boundary 540 defined during method 400A provides sufficient protection for the at-risk portion 640 of surface 610. For example, visual representations of at-risk portion 640, projection 642, or both may be displayed via graphical user interface 500.

FIG. 3E illustrates one embodiment of the guidance process 414 in greater detail by illustrating a method 414a for providing guidance information. At a process 480, a three-dimensional zone (e.g., cone-shaped projection 642) is generated as extending from instrument exit point 635 toward target 620. At a process 482, a two-dimensional projection of the three-dimensional zone is displayed with the image data 510. As depicted in FIG. 5C, a two-dimensional projection region 650 of the three-dimensional zone (e.g., cone 642) is provided in overlay on the two-dimensional image data 510 depicting the target 620. At a process 484, with the projection 650 as a guide, the user may generate curve 652 as described in processes 420 and 430. Curve 652 may be drawn to extend within and optionally, beyond the region 650 to define a boundary of the at-risk portion 640. As previously described, additional curves may be drawn in additional slices of two-dimensional image data 510 to generate multiple curves used to generate the anatomical border 540. In some embodiments, pixels in the at-risk area may be displayed in a different shade, color, or semi-transparent color overlay. Guidance may be turned on or off, either automatically, by user selection, or by a combination. Additionally or alternately, an indicator of whether anatomical boundary 540 fully protects at-risk portion 640 may be displayed via graphical user interface 500 or otherwise communicated to the operator.

As depicted in FIG. 5B, one factor that may give rise to varying levels of risk is uncertainty associated with the medical procedure (e.g., uncertainty in the location of exit point 635, uncertainty in the location of target 620, or both). Other factors that may give rise to varying levels of risk include the distance between surface 610 and exit, point 635; locations further from exit point 635 are generally at a lower risk than closer locations.

During a planning procedure, a safety score may be computed and provided to the operator that indicates the likelihood that the instrument will breach the boundary 540. Based on the score, the planned navigational path may be adjusted or revised to achieve a safer route. A variety of paths with different, safety scores may be provided to the operator for selection.

Referring again to FIG. 3D, additional guidance information and visualization aides may be provided at the process 422. FIG. 3F illustrates one embodiment of the guidance process 422 in greater detail by illustrating a method 422a for providing guidance information. At a process 486, a projection or shadow of curve 520 may be displayed in other planes of image data 510, where curve 520 otherwise would not be displayed (e.g., in CT slices of image data 510 other than the slice that includes curve 520). Accordingly, the projection or shadow of curve 520 provides guidance in the form of a reminder to the operator of the characteristics of curve 520 (e.g., the starting point, ending point, length, etc.) when defining curve 530. Absent such a reminder, the operator may inadvertently define curve 530 with significantly different characteristics than curve 520 (e.g., a significantly different start position, end position, or length). In such cases, anatomical boundary 540 may have an irregular shape or otherwise may not correspond to the desired anatomical boundary.

At a process 488, guidance information may include a starting point and an ending point of the first curve. In some embodiments, anatomical boundary 540 may also have an irregular shape when curve 530 is inadvertently flipped relative to curve 520 (e.g., when the respective start and end points are on opposite ends of the curves). For example, anatomical boundary 540 may have a twisted shape when the directions are flipped. Accordingly, guidance information may be displayed to indicate which direction curve 530 should be oriented to match curve 520. For example, with respect to the projection or shadow of curve 520 discussed above (or, analogously, the projection of anatomical boundary 540), a starting point may be displayed in visually distinguishable manner from the end point (e.g., using different colors, patterns, textures, etc.).

Referring again to FIG. 3D, instrument or boundary adjustment guidance information may be provided at the process 452. FIG. 3G illustrates one embodiment of the guidance process 452 in greater detail by illustrating a method 452a for providing guidance information. For example, at an optional process 490, a projection or shadow of anatomical boundary 540 may be extrapolated and displayed in regions outside the current range of anatomical boundary 540 to provide guidance to the operator when extending the range of anatomical boundary 540. In some embodiments, the projection or shadow of anatomical boundary 540 may be displayed in a visually distinguishable manner from anatomical boundary 540 itself (e.g., using different colors, patterns, textures, etc.) to alert the operator to whether the currently displayed cross section is within or outside of the current range of anatomical boundary 540. As previously described, the plan for the biopsy procedure, including the anatomical boundary 540 may be saved and used by the control system to provide automated navigation or operator navigation assistance of a medical instrument to perform the biopsy procedure. During navigation, the boundary 540 may be displayed with a three-dimensional anatomic model of the anatomic region (e.g., view 512), with an endoluminal view, or with other anatomical views presented on a user display. The boundary 540 may also or alternatively be displayed with (e.g., overlaid on) registered images from other imaging technology such as fluoroscopic images obtained during a medical procedure.

At an optional process 491, suggested deployment locations for a medical instrument may be provided. For example, during a registration procedure to register the three-dimensional model to the patient anatomy, a point gathering medical instrument may be used to touch a recommended cloud of points in the patient anatomy. The recommended cloud of points may be determined based on their location relative to the boundary 540. For example a point may be recommended only if it is within a threshold distance from the boundary 540. Similarly, during the biopsy procedure, recommended biopsy locations may be determined based on their location relative to the boundary 540. For example a biopsy point may be recommended only if it is within a threshold distance from the boundary 540.

At an optional process 492, during a medical procedure, the position and orientation of the medical instrument relative to the anatomical boundary 540 may be monitored. A distance between the medical instrument and the anatomical boundary 540 may be measured, for example, from the distal end portion of the instrument or from a portion of the instrument that is closest to the anatomical boundary 540. At a process 493, when the distance between the instrument and the anatomical boundary 540 becomes less than a predetermined threshold distance value, an indicator may be provided to an operator. For example, a visual indicator on the graphical user interface 500 may be provided in the form of a color change, textual alert, highlighted instrument, highlighted boundary 540, or other visual warning signal. Indicators may also be provided in the form of audible, haptic, or other operator-perceptible signals. Additionally or alternatively, at a process 494, the control system 112 may monitor the distance and slow the instrument speed or stop it completely as it approaches the surface corresponding to the boundary 540. Additionally or alternatively, at a process 495, the operator may provide a user input (e.g., pressing a button) that will aim the distal end of the medical instrument away from the surface corresponding to the boundary 540. Additionally or alternatively, the distance based-indicator may be used in a planning procedure with a virtual medical instrument.

One or more elements in embodiments of this disclosure may be implemented in software to execute on a processor of a computer system such as control processing system. When implemented in software, the elements of the embodiments of the invention are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In one embodiment, the control system supports wireless communication protocols such as Bluetooth, IrDA, HomeRF, IEEE 802.11, DECT, and Wireless Telemetry.

Medical tools that may be delivered through the flexible elongate devices or catheters disclosed herein may include, for example, image capture probes, biopsy instruments, laser ablation fibers, and/or other surgical, diagnostic, or therapeutic tools. Medical tools may include end effectors having a single working member such as a scalpel, a blunt blade, an optical fiber, an electrode, and/or the like. Other end effectors may include, for example, forceps, graspers, scissors, clip appliers, and/or the like. Other end effectors may further include electrically activated end effectors such as electrosurgical electrodes, transducers, sensors, and/or the like. Medical tools may include image capture probes that include a stereoscopic or monoscopic camera for capturing images (including video images). Medical tools may additionally house cables, linkages, or other actuation controls (not shown) that extend between its proximal and distal ends to controllably bend the distal end of medical instrument 304. Steerable instruments are described in detail in U.S. Pat. No. 7,316,681 (filed on Oct. 4, 2005) (disclosing “Articulated Surgical Instrument for Performing Minimally Invasive Surgery with Enhanced Dexterity and Sensitivity”) and U.S. patent application Ser. No. 12/286,644 (filed Sep. 30, 2008) (disclosing “Passive Preload and Capstan Drive for Surgical Instruments”), which are incorporated by reference herein in their entireties.

The systems described herein may be suited for navigation and treatment of anatomic tissues, via natural or surgically created connected passageways, in any of a variety of anatomic systems, including the lung, colon, the intestines, the kidneys and kidney calices, the brain, the heart, the circulatory system including vasculature, and/or the like.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the embodiments of the invention are not described with reference to any particular programming language. It will be appreciated that a variety of programming languages may be used to implement the teachings of the invention as described herein.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

1. A medical system comprising:
a display system;
a user input device;
a medical instrument;
a manipulator assembly configured to support and operate the medical instrument; and
a control system communicatively coupled to at least the display system the user input device, and the manipulator assembly, the control system configured to:
display image data corresponding to a three-dimensional anatomical region via the display system;
receive a first user input to generate a first curve in the three-dimensional anatomical region via the user input device;
receive a second user input to generate a second curve in the three-dimensional anatomical region via the user input device;
determine an anatomical boundary bounded by the first curve and the second curve, the anatomical boundary indicating a surface of an anatomical structure in the three-dimensional anatomical region; and
control the manipulator assembly to operate medical instrument using the determined anatomical boundary to limit movement of the medical instrument.

2-4. (canceled)

5. The medical system of claim 1, wherein the control system is further configured to:
receive a third user input to generate a third curve in the three-dimensional anatomical region via the user input device;
adjust the anatomical boundary to be bounded by the first curve, the second curve, and the third curve; and
display the adjusted anatomical boundary with the image data via the display system.

6-9. (canceled)

10. The medical system of claim 1, wherein the control system is configured to determine the anatomical boundary based on an intensity gradient associated with the image data.

11. The medical system of claim 1, wherein the control system is further configured to apply computer vision to the image data to identify a candidate anatomical boundary, and wherein the anatomical boundary is snapped to the candidate anatomical boundary.

12. The medical system of claim 1, wherein the control system is further configured to display guidance information via the display system during placement of the second curve.

13-14. (canceled)

15. The medical system of claim 12, wherein the guidance information includes an extrapolated projection of the anatomical boundary in regions outside of a current range of the anatomical boundary.

16. The medical system of claim 1, wherein the control system is further configured to:
display the anatomical boundary overlaid on an anatomic model derived from the image data via the display system; and
deform the anatomical boundary to conform with deformations of the anatomic model based on movement of a patient anatomy.

17. (canceled)

18. The medical system of claim 1, wherein the control system is further configured to:
display the anatomical boundary overlaid on fluoroscopic image data obtained during a patient procedure.

19. The medical system of claim 1, wherein the control system is further configured to:
receive a third user input while a medical instrument is located within the three-dimensional anatomical region and
responsive to the third user input, direct an orientation of a distal end of the medical instrument away from the anatomical boundary.

20. The medical system of claim 1, wherein the control system is further configured to determine a distance between a distal end of a virtual medical instrument and the anatomical boundary.

21. The medical system of claim 1, wherein the control system is further configured to:
determine a distance between a distal end of a medical instrument and the anatomical boundary.

22. The medical system of claim 21 wherein the control system is further configured to:
provide a visual, audible, or haptic indicator when the distance between the distal end of the medical instrument and the anatomical boundary is less than a predetermined threshold distance.

23. The medical system of claim 21 wherein the control system is further configured to:
alter an advancement speed of the medical instrument based on the determined distance.

24. The medical system of claim 1 wherein the control system is further configured to:
provide one or more suggested deployment locations for a medical instrument, wherein the one or more suggested deployment locations are located at least a threshold distance from the anatomical boundary.

25. The medical system of claim 1, wherein the control system is further configured to:
generate a three-dimensional zone based on an instrument exit point and a target.

26. The medical system of claim 25, wherein the control system is further configured to:
display a two-dimensional projection of the zone with the image data to determine an at-risk portion of the surface based on an intersection between the surface and the zone.

27. The medical system of claim 26, wherein the first curve is generated at least partially along the intersection to mark the at-risk portion relative to the image data.

28. A method of planning a medical procedure, the method comprising:
displaying, via a display system, image data corresponding to a three-dimensional anatomical region;
receiving, via a user input device, a plurality of user inputs to generate a plurality of curves in the three-dimensional anatomical region;
determining from the plurality of curves an anatomical boundary, the anatomical boundary demarcating a portion of interest of the three-dimensional anatomical region;
displaying, via the display system, the anatomical boundary overlaid on the image data; and
determine a distance between a distal end of a medical instrument and the anatomical boundary.

29-42. (canceled)

43. The method of claim 28, further comprising:
directing an orientation of a distal end of the medical instrument away from the anatomical boundary based on the determined distance.

44-49. (canceled)

50. A non-transitory machine-readable medium comprising a plurality of machine readable instructions which when executed by one or more processors associated with a planning workstation are adapted to cause the one or more processors to perform a method comprising:
displaying, via a display system, CT image data corresponding to a lung;
receiving, via a user input device, a plurality of user inputs to generate a plurality of curves in different slices of the CT image data;
interpolating among the plurality of curves to determine an anatomical boundary indicating a location of a pleura of the lung in the CT image data;
displaying, via the display system, the anatomical boundary overlaid on the CT image data; and
providing a variety of planned navigation paths for navigating a medical instrument based on different likelihoods that the medical instrument will breach the anatomical boundary.
